(19) United States
(12) Patent Application Publication
(10) Pub. No.: US 2026/0060562 A1
(43) Pub. Date: Mar. 5, 2026

(54) Laser-Based Ambient Sensing for Mobile Computing Devices

(71) Applicant: Google LLC, Mountain View, CA (US)

(72) Inventors: Octavio Ponce Madrigal, Mountain View, CA (US); Michal Matuszak, San Jose, CA (US)

(21) Appl. No.: 18/816,781

(22) Filed: Aug. 27, 2024

(51) Int. Cl. A61B 5/024; A61B 5/00; A61B 5/08; H04N 23/67

(52) U.S. Cl. CPC A61B 5/02416; A61B 5/0816; A61B 5/6898; A61B 5/7267; H04N 23/671

(57) ABSTRACT

Computing systems, computing devices, and computer-implemented methods are provided. In one aspect, a mobile computing device includes a laser-based sensor such as a laser detect auto-focus sensor of an image capture assembly having an image capture device. The mobile computing device further includes one or more computing devices configured to perform one or more operations. For instance, the operations may include generating, with one or more laser-based sensors of a mobile computing device over a period of time, sensor data indicative of a distance between the mobile computing device and at least one surface, and generating, with one or more machine-learned models based on the sensor data, ambient sensing data including at least one biometric associated with a user of the mobile computing device.

### FIELD

The present disclosure relates generally to mobile computing devices.

### BACKGROUND

Computing devices capable of monitoring and detecting health-related information associated with a user of the computing device may track the user's activities and/or biometrics using a variety of sensors. Data captured from these sensors may be analyzed in order to provide the user with information such as, for instance, an estimation of their skin temperature, how far they walked in a day, their heart rate, how much time they spent sleeping, and the like. As demand for additional on-device capabilities increases, there is a need for re-purposing existing components to provide additional on-device sensing functionalities.

### BRIEF DESCRIPTION OF THE DRAWINGS

Detailed discussion of embodiments directed to one of ordinary skill in the art are set forth in the specification, which makes reference to the appended figures, in which:

FIG. 1 depicts a front view of an example mobile computing device according to example embodiments of the present disclosure;

FIG. 2 depicts a rear view of an example mobile computing device according to example embodiments of the present disclosure;

FIG. 3 depicts a side view of an example mobile computing device according to example embodiments of the present disclosure;

FIG. 4 depicts a rear perspective view of an example mobile computing device according to example embodiments of the present disclosure;

FIG. 5 depicts a front view of an example mobile computing device according to example embodiments of the present disclosure;

FIG. 6 depicts a rear view of an example mobile computing device according to example embodiments of the present disclosure;

FIG. 7 depicts a block diagram of an example mobile computing device according to example embodiments of the present disclosure;

FIG. 8 depicts a flow-chart diagram of an example method according to example embodiments of the present disclosure;

FIGS. 9A and 9B depict systems for ambient sensing using a laser-based sensor of a user computing device according to example embodiments of the present disclosure;

FIG. 10A depicts a block diagram of a computing environment including an ambident sensing system according to example embodiments of the present disclosure;

FIG. 10B depicts a graphical illustration of processing LDAF sensor data by filtering and binning to determine respiration rate according to example embodiments of the present disclosure;

FIG. 11 depicts a graphical illustration of processing LDAF sensor data by filtering and binning to determine respiration rate according to example embodiments of the present disclosure;

FIG. 12 depicts a graphical illustration of processing LDAF sensor data by filtering and binning to determine respiration rate according to example embodiments of the present disclosure;

FIG. 13 depicts a block diagram of a processing pipeline for processing LDAF sensor data for multiple applications according to example embodiments of the present disclosure; and FIG. 14 depicts a block diagram of a processing pipeline for processing LDAF sensor data for a meditation application according to example embodiments of the present disclosure;

Repeat use of reference characters in the present specification and drawings is intended to represent the same and/or analogous features or elements of the present invention.

### DETAILED DESCRIPTION

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations may be made to the embodiments without departing from the scope or spirit of the present disclosure. For instance, features illustrated or described as part of one embodiment may be used with another embodiment to yield a still further embodiment. Thus, it is intended that aspects of the present disclosure cover such modifications and variations. Furthermore, it should be understood that the drawings are intended to represent structures for purposes of identification and description and are not intended to represent the structures to physical scale.

As used herein, the terms “first,” “second,” and “third” may be used interchangeably to distinguish one component from another and are not intended to signify location or importance of the individual components. The terms “includes” and “including” are intended to be inclusive in a manner similar to the term “comprising. ” Similarly, the term “or” is generally intended to be inclusive (e.g., “A or B” is intended to mean “A or B or both”). The term “at least one of” in the context of, e.g., “at least one of A, B, and C” refers to only A, only B, only C, or any combination of A, B, and C. In addition, here and throughout the specification and claims, range limitations may be combined and/or interchanged. Such ranges are identified and include all the sub-ranges contained therein unless context or language indicates otherwise. For example, all ranges disclosed herein are inclusive of the endpoints, and the endpoints are independently combinable with each other. The singular forms “a,” “an,” and “the” include plural references unless the context clearly dictates otherwise.

Approximating language, as used herein throughout the specification and claims, may be applied to modify any quantitative representation that could permissibly vary without resulting in a change in the basic function to which it is related. Accordingly, a value modified by a term or terms, such as “generally,” “about,” “approximately,” and “substantially,” are not to be limited to the precise value specified. In at least some instances, the approximating language may correspond to the precision of an instrument for measuring the value, or the precision of the methods or machines for constructing or manufacturing the components and/or systems. For example, the approximating language may refer to being within a 10 percent margin, i.e., including values within ten percent greater or less than the stated value. In this regard, for example, when used in the context of an angle or direction, such terms include within ten degrees greater or less than the stated angle or direction, e.g., “generally vertical” includes forming an angle of up to ten degrees in any direction, e.g., clockwise or counterclockwise, with the vertical direction V.

The word “exemplary” is used herein to mean “serving as an example, instance, or illustration. ” In addition, references to “an embodiment” or “one embodiment” do not necessarily refer to the same embodiment, although it may. Any implementation described herein as “exemplary” or “an embodiment” is not necessarily to be construed as preferred or advantageous over other implementations. Moreover, each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

Relative terms such as “below” or “above” or “upper” or “lower” or “horizontal” or “lateral” or “vertical” may be used herein to describe a relationship of one element, layer or region to another element, layer or region as illustrated in the figures. It will be understood that these terms are intended to encompass different orientations of the device in addition to the orientation depicted in the figures. Furthermore, unless otherwise defined, all terms (including technical and scientific terms) used herein have the same meaning as commonly understood by one of ordinary skill in the art to which this invention belongs. It will be further understood that terms used herein should be interpreted as having a meaning that is consistent with their meaning in the context of this specification and the relevant art and will not be interpreted in an idealized or overly formal sense unless expressly so defined herein.

In the drawings and specification, there have been disclosed typical embodiments and, although specific terms are employed, they are used in a generic and descriptive sense only and not for purposes of limitation of the scope set forth in the following claims. Furthermore, like numbers refer to like elements throughout. Thus, the same or similar numbers may be described with reference to other drawings even if they are neither mentioned nor described in the corresponding drawing. Also, elements that are not denoted by reference numbers may be described with reference to other drawings.

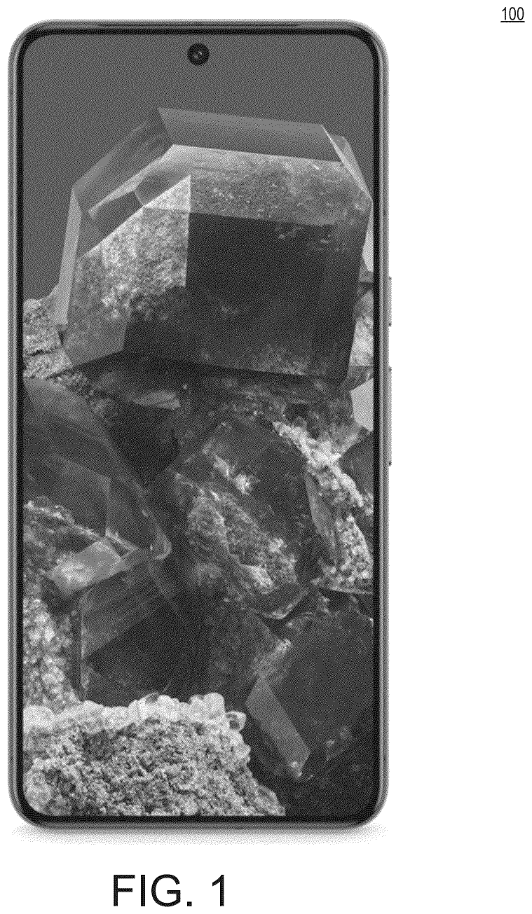

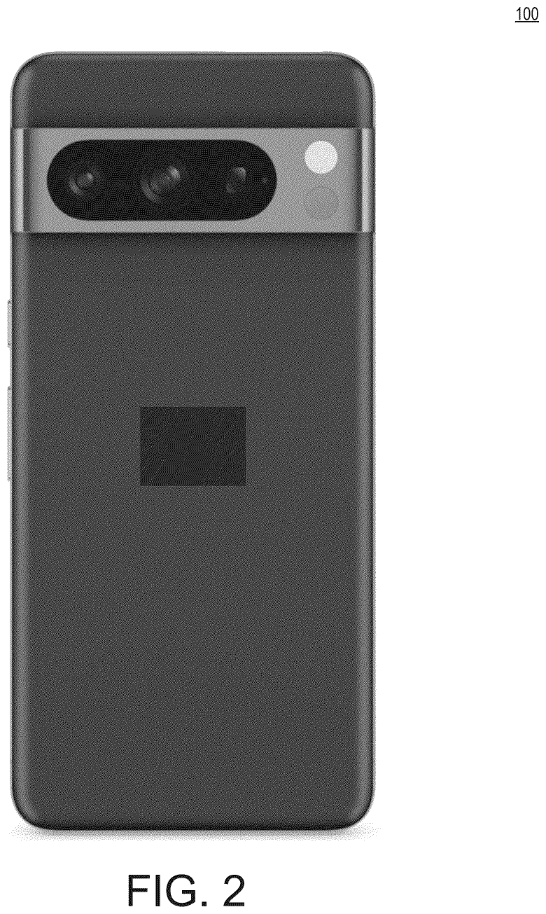

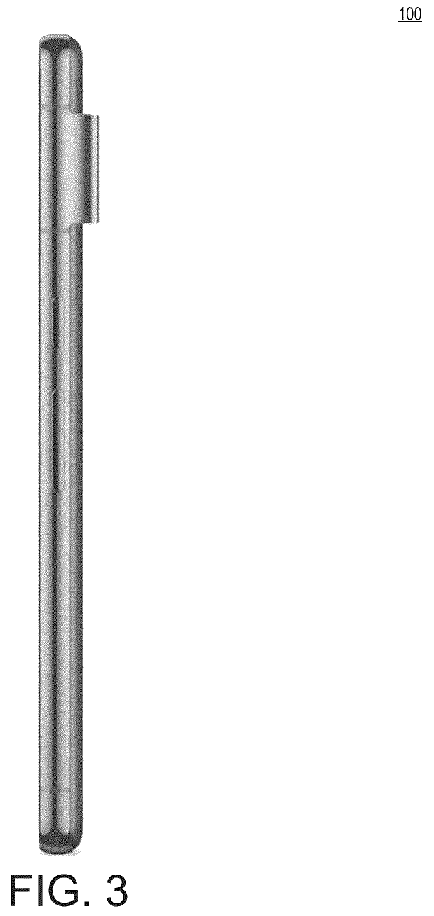

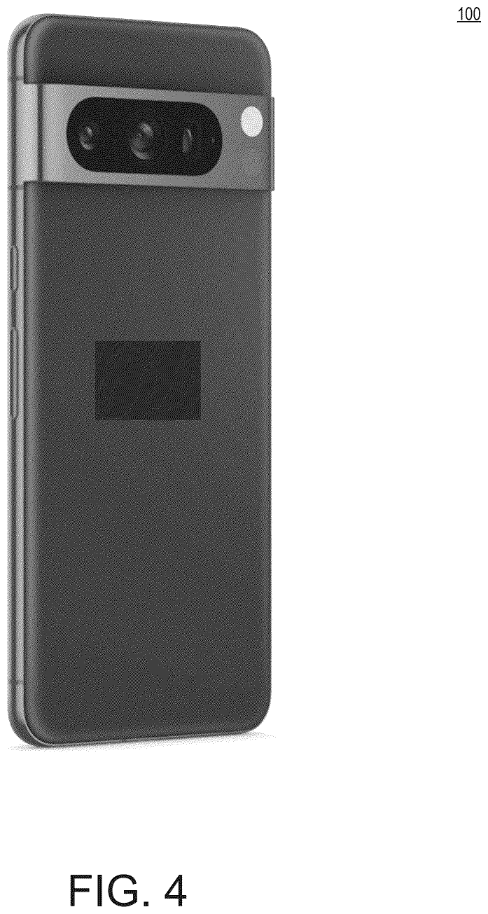

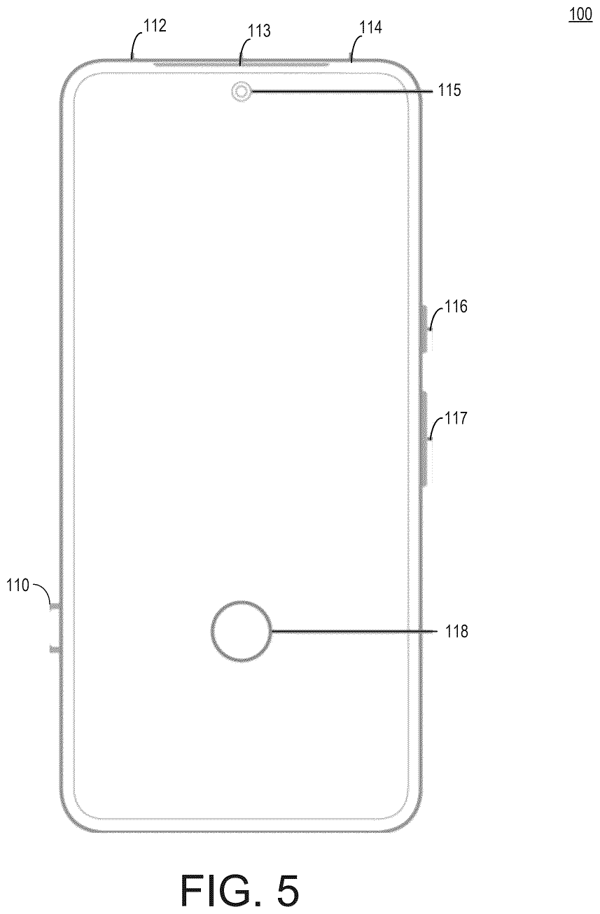

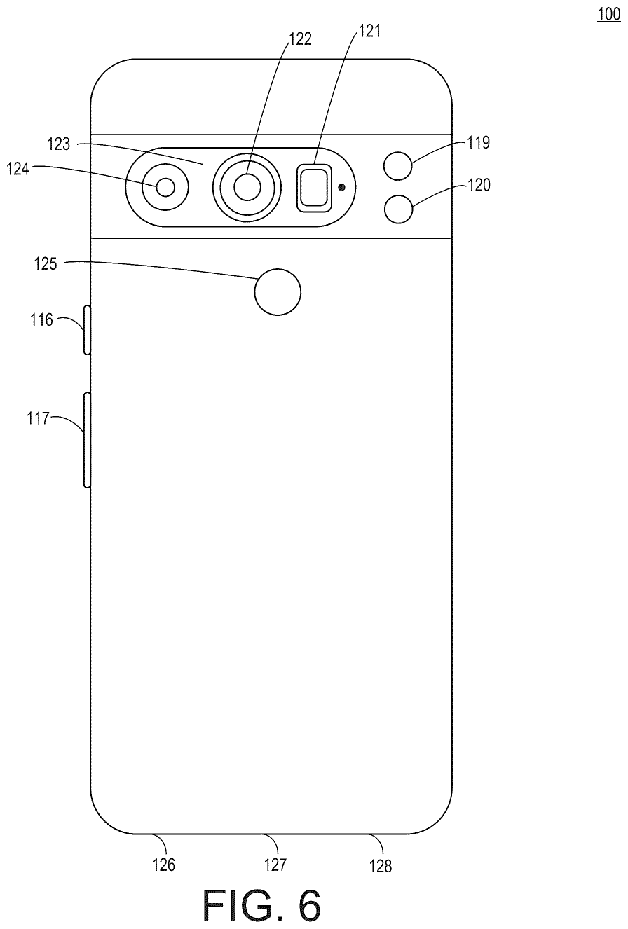

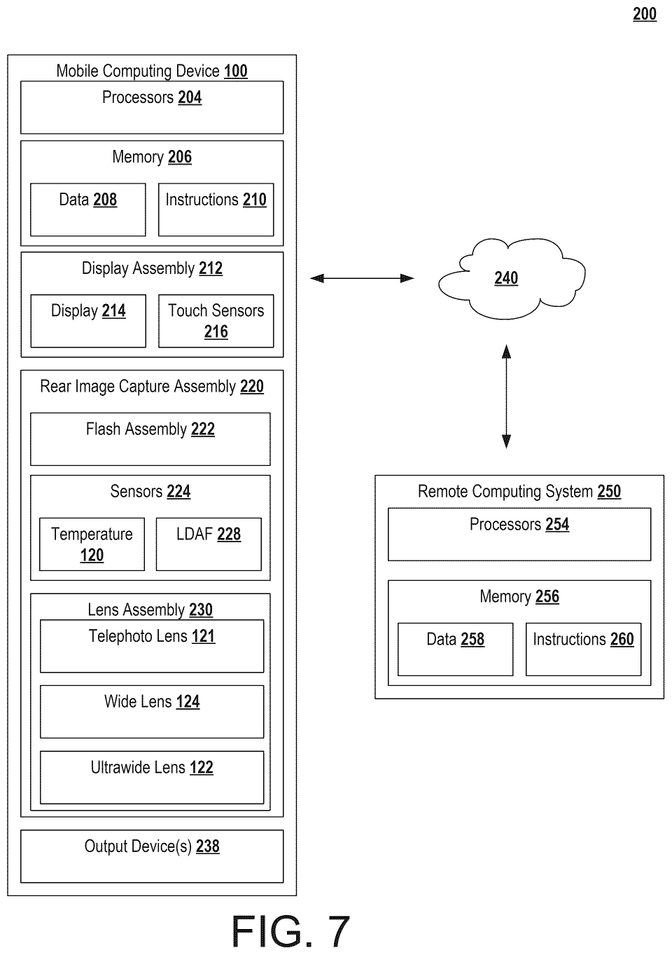

FIG. 1-7 depict an example mobile computing device 100 according to example embodiments of the present disclosure. While much of the disclosure describes a smart phone as an example mobile computing device, it will be appreciated that a mobile computing device can include any suitable computing device such as a smart watch, a mixed or augmented reality headset, smart glasses, earbuds, and the like. More particularly, FIG. 1 depicts a front view of the example mobile computing device. FIG. 2 depicts a rear view of the example mobile computing device. FIG. 3 depicts a side view of the example mobile computing device. FIG. 4 depicts a rear perspective view of the example mobile computing device. FIG. 5 depicts a front view of the example mobile computing device. FIG. 6 depicts a rear view of the example mobile computing device. FIG. 7 depicts a block diagram of example components of the example mobile computing device. It should be understood that FIG. 1-7 depict the example mobile computing device and its various components for purposes of illustration and discussion. Those having ordinary skill in the art, using the disclosures provided herein, will appreciate that example aspects of the present disclosure may be implemented by any suitable computing device, such as, by way of non-limiting example, a mobile tablet device, a wearable computing device, and the like.

Referring now to FIG. 1-7, the mobile computing device may include a housing. The housing may include any suitable material, such as aluminum, titanium, and the like. As will be discussed in greater detail below, the housing may define a back surface (e.g., back side), a top surface (e.g., top side), a bottom surface (e.g., bottom side), and one or more side surfaces (e.g., left side, right side, etc.) of the mobile computing device. The housing may further define a cavity (e.g., internal volume) (not shown) in which one or more electronic components (e.g., disposed on printed circuit boards) are disposed. For instance, the mobile computing device may include a printed circuit board (e.g., flexible printed circuit board) (not shown) disposed within the cavity. The mobile computing device may further include a battery (not shown) that is disposed within the cavity defined by the housing. Furthermore, the mobile computing device may also include one or more internal temperature sensors (not shown) within the cavity that are configured to obtain internal temperature data indicative of an internal temperature of the mobile computing device.

The mobile computing device may include a display assembly 212. The display assembly may define a front surface (e.g., front side) of the mobile computing device. The display assembly may be configured to display content (e.g., time, date, biometric, notifications, etc.) for viewing by the user and to receive inputs from the user. Furthermore, as discussed below, the display assembly may be a touch-sensitive display assembly that is sensitive to the touch of a user object (e.g., finger, stylus, and the like). The touch-sensitive display assembly may serve to implement, for instance, a virtual keyboard. The touch-sensitive display assembly can include one or more touch sensors 216.

More particularly, in some examples, the display assembly may include a display 214. The display may include a plurality of pixels. For instance, in some examples, the display may include an organic light-emitting diode (OLED) display. It should be understood, however, that the display may include any suitable display without deviating from the scope of the present disclosure. In some examples, the display may be an “always-on” display operable to display content to the user in a quickly accessible way (e.g., “At a Glance”). In particular, in some examples, content is displayed on the display even when the user is not explicitly interacting with the computing device. In this manner, users may quickly access information by viewing content and performing actions without needing to invoke the computing system (e.g., performing “wake up”functions to activate the computing system).

The display assembly may further include a display cover positioned on the housing such that the display cover is positioned on top of the display. In this manner, the display cover may protect the display from being damaged (e.g., scratched or cracked). In some examples, the display assembly may include a seal positioned between the housing and the display cover. For instance, a first surface of the seal may contact the housing and a second surface of the seal may contact the display cover. In this manner, the seal between the housing and the display cover may prevent a liquid (e.g., water) from entering the cavity defined by the housing. It should be understood that the display cover may be optically transparent so that the user may view information being displayed on the display. For instance, in some examples, the display cover may include a glass material. It should be understood, however, that the display cover may include any suitable optically transparent material.

The display assembly may further include one or more touch sensors 216 operable to detect one or more inputs (e.g., touch inputs) provided by the user touching the display assembly (e.g., display cover). In this manner, the display assembly may be a touch-sensitive display assembly. In some examples, one or more of the touch sensors may include a capacitive sensor whose capacitance changes when a touch input is provided at a location on the display cover that corresponds to the capacitive sensor. It should be understood, however, that the touch sensors may include any suitable type of sensor configured to detect a touch input provided by the user touching the display cover.

The display assembly may further include a controller. The controller may include one or more processors and a memory. The processor(s) may be communicatively coupled to the plurality of touch sensors. In this manner, the processor(s) may receive signals from the plurality of touch sensors. Furthermore, the memory may store instructions that, when executed by the processor(s), cause the processor(s) to process the signals received from the plurality of touch sensors and perform one or more actions based on the location at which the touch input was provided. For instance, in some examples, the processor(s) may be configured to update the content displayed by the display assembly, specifically a display layer thereof.

The mobile computing device may include one or more image capture assemblies. For instance, the mobile computing device may include a front image capture assembly on/within the front surface of the mobile computing device. The front image capture assembly may include, for instance, a front-facing camera 115 operable to capture images and/or videos. In some examples, the front image capture assembly may be operable to implement a variety of image capture-related tasks, such as autofocus of an aperture and/or lens and the like. It should be understood that the front image capture assembly may include any suitable image capture device without deviating from the scope of the present disclosure.

The mobile computing device may further include a rear image capture assembly 220 on a rear/back surface of the mobile computing device. In some examples, such as that depicted in FIG. 1-7, the rear image capture assembly may include a plurality of image capture devices (e.g., lens assembly) operable to capture images and/or videos. For instance, in some examples, the rear image capture assembly may include a wide camera lens 124, an ultrawide camera lens 122, and a telephoto camera lens 121. The rear image capture assembly may also include a flash device and/or flash assembly 222, such as an LED flash 119. In some examples, the rear image capture assembly may be operable to implement a variety of image capture-related tasks, such as auto focus of an aperture and/or lens, lens correction, zoom, optical and/or electronic image stabilization, and the like. By way of non-limiting example, as described below, the rear image capture assembly may include a laser detect auto-focus (LDAF) system 228 operable to automatically focus one or more apertures/lenses for the mobile computing device.

As will be discussed in greater detail below, the mobile computing device may include (and receive data from) one or more sensors 224. The one or more sensors may be housed in the housing. The one or more sensors may include one or more image sensors (e.g., image capture devices discussed above), one or more LIDAR sensors, one or more audio sensors (e.g., microphone(s)), one or more inertial sensors (e.g., inertial measurement unit(s) (IMU(s))), one or more biometric sensors (e.g., heart rate sensor(s), pulse sensor(s), retinal sensor(s), fingerprint sensor(s) 118, etc.), one or more touch sensors (e.g., conductive touch sensor(s), mechanical touch sensor(s)) (discussed above), one or more infrared (IR) sensors, one or more optical sensors, one or more location sensors (e.g., GPS), one or more temperature sensors 120, and/or one or more other sensors. The mobile computing device includes an example top microphone 114 and a bottom microphone 128. The one or more sensors may be used to obtain data associated with the user's environment (e.g., an image of a user's environment, a recording of the environment, a location of the user, an authentication of the user, a temperature of the user and/or an object in the environment, etc.). It should be understood that the one or more sensors may include any suitable sensor without deviating from the scope of the present disclosure.

The mobile computing device may further include one or more buttons and/or ports. For instance, in some examples, the mobile computing device may include a power port 127 operable to provide charge to the battery and a USB-C port 127. The mobile computing device may also include one or more volume buttons 117 operable to control a volume of audio output by one or more speakers such as top speaker 113 and bottom speaker 116. The mobile computing device may also include a power button 116 operable to control a power state (e.g., “ON,” “OFF,” “IDLE,” “STANDBY,” etc.) of the mobile computing device. It should be understood that the mobile computing device may include any suitable button and/or port without deviating from the scope of the present disclosure.

The mobile computing device may be operable to communicate with remote computing systems and devices and/or third-party computing systems and devices over a variety of telecommunications networks. For instance, the mobile computing device may include a Subscriber Identity Module (SIM) card insertable in SIM card tray 110, which, in conjunction with one or more antennas (e.g., mmWave antenna beneath cover 112), allows the mobile computing device to communicate over one or more telecommunications networks, such as a cellular network and the like. The mobile computing device may also be operable to connect to wireless networks, such as local area networks, Wi-Fi networks, and the like. Even further, the mobile computing device may include Near Field Communication (NFC) components operable to provide NFC capabilities to the mobile computing device. Mobile computing device 100 includes an NFC antenna 125.

In some examples, the mobile computing device may include one or more output devices 238. For instance, as noted above, the one or more output devices may include the display. The one or more output devices may further include one or more speakers. In this manner, the mobile computing device may emit audible noises (e.g., alarm, voice automated messages, audio, etc.) for the user. The one or more output devices may further include one or more haptic devices operable to provide one or more haptic notifications (e.g., vibratory notifications) to the user. It should be appreciated that the mobile computing device may include any suitable output device without deviating from the scope of the present disclosure.

The mobile computing device may further include one or more processors 204 and a memory 206. The one or more processors may include any suitable processing device (e.g., a processor core, a microprocessor, an application specific integrated circuit (AISC), a field programmable gate array (FPGA), a microcontroller, etc.). In some examples, the one or more processors may be communicatively coupled to the one or more sensors. For instance, the one or more processors may be communicatively coupled to the one or more sensors via a data interface (e.g., data bus). In this manner, the one or more processors may obtain data from the one or more sensors 224. In some examples, the one or more processors may determine one or more metrics, such as a proximity metric, a biometric, and the like, based on the data obtained from the one or more sensors. The memory may include one or more non-transitory computer-readable storage media, such as random-access memory (RAM), read-only memory (ROM), electronically erasable programmable ready-only memory (EEPROM), erasable programmable read-only memory (EPROM), flash memory devices, and combinations thereof. The memory may store data 208 and instructions 210 that, when executed by the one or more processors, cause the one or more processors to perform one or more operations, such as any of the operations disclosed herein.

As noted above, the mobile computing device may be part of a computing system or computing environment 200, which may be operable to implement any of the methods and/or operations disclosed herein. The computing system may include the mobile computing device 100 and a remote computing system 250. The mobile computing device may be communicatively coupled to the remote computing system over a network 240. As noted above, the network may be any type of communications network, such as a local area network (e.g., intranet), wide area network (e.g., Internet), or some combination thereof and may include any number of wired or wireless links. In general, communication over the network may be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

The remote computing system 250 may include one or more processors 254 and a memory 256. The one or more processors may be any suitable processing device (e.g., a processor core, a microprocessor, an ASIC, an FPGA, a controller, a microcontroller, etc.) and may be one processor or a plurality of processors that are operatively connected. The memory may include one or more non-transitory computer-readable storage medium(s), such as RAM, ROM, EEPROM, EPROM, flash memory devices, magnetic disks, etc., and combinations thereof. The memory may store data 258 and instructions 260 which are executed by the processor to cause the remote computing system to perform operations, such as any of the operations described herein. In this manner, the computing system may be operable to implement any of the methods described herein.

In some examples, the remote computing system may include or may otherwise be implemented by one or more computing devices. In instances in which the remote computing system includes plural server computing devices, such server computing devices may operate according to sequential computing architectures, parallel computing architectures, or some combination thereof.

Furthermore, the computing system may include one or more machine-learned models. For instance, the machine-learned models may be or may otherwise include various machine-learned models such as neural networks (e.g., deep neural networks) or other types of machine-learned models, including non-linear models and/or linear models. Neural networks may include feed-forward neural networks, recurrent neural networks (e.g., long short-term memory recurrent neural networks), convolutional neural networks or other forms of neural networks.

In some examples, the one or more machine-learned models may be received from the server computing system over the network, stored in the mobile computing device memory, and then used or otherwise implemented by the one or more processors. In some examples, the mobile computing device may implement multiple parallel instances of a single machine-learned model (e.g., to perform parallel machine-learned model processing across multiple instances of input data and/or detected features).

More particularly, the one or more machine-learned models may include one or more detection models, one or more classification models, one or more segmentation models, one or more augmentation models, one or more generative models, one or more natural language processing models, one or more optical character recognition models, and/or one or more other machine-learned models. The one or more machine-learned models may include one or more transformer models. The one or more machine-learned models may include one or more neural radiance field models, one or more diffusion models, and/or one or more autoregressive language models.

The one or more machine-learned models may be utilized to detect one or more object features. The detected object features may be classified and/or embedded. The classification and/or the embedding may then be utilized to perform a search to determine one or more search results. Alternatively and/or additionally, the one or more detected features may be utilized to determine an indicator (e.g., a user interface element that indicates a detected feature) is to be provided to indicate a feature has been detected. The user may then select the indicator to cause a feature classification, embedding, and/or search to be performed. In some implementations, the classification, the embedding, and/or the searching may be performed before the indicator is selected.

In some examples, the one or more machine-learned models may process image data, text data, audio data, and/or latent encoding data to generate output data that may include image data, text data, audio data, and/or latent encoding data. The one or more machine-learned models may perform optical character recognition, natural language processing, image classification, object classification, text classification, audio classification, context determination, action prediction, image correction, image augmentation, text augmentation, sentiment analysis, object detection, error detection, inpainting, video stabilization, audio correction, audio augmentation, and/or data segmentation (e.g., mask based segmentation).

Additionally and/or alternatively, one or more machine-learned models may be included in or otherwise stored and implemented by the server computing system that communicates with the mobile computing device according to a client-server relationship. For instance, the machine-learned models may be implemented by the server computing system as a portion of a web service (e.g., a viewfinder service, a visual search service, an image processing service, an ambient computing service, and/or an overlay application service). Thus, one or more models may be stored and implemented at the mobile computing device and/or one or more models may be stored and implemented at the server computing system.

The technology discussed herein refers to sensors and other computer-based systems, as well as actions taken, and information sent to and from such systems. One of ordinary skill in the art will recognize that the inherent flexibility of computer-based systems allows for a great variety of possible configurations, combinations, and divisions of tasks and functionality between and among components. For instance, server processes discussed herein may be implemented using a single server or multiple servers working in combination. Databases and applications may be implemented on a single system or distributed across multiple systems. Distributed components may operate sequentially or in parallel.

FIG. 1-7 and the corresponding discussion illustrate and describe an example mobile communication device for purposes of illustration and discussion. It should be understood, however, that any suitable mobile computing device may be used without deviating from the scope of the present disclosure.

As noted above, the rear image capture assembly of the example mobile computing device discussed herein may include a laser detect auto-focus (LDAF) system that provides autofocus capabilities to the rear image capture assembly. The LDAF sensor may be an optical sensor, such as a laser-based optical sensor. The LDAF sensor may be part of the rear image capture assembly and may be configured to assist in focusing the lenses and/or apertures of the one or more image capture devices by determining one or more proximity metrics for a subject and/or target. It should be understood that the terms “subject” and “target” may be used interchangeably. For instance, LDAF sensors according to the present disclosure are operable to estimate distances in millimeter precision. For instance, by way of non-limiting example, an LDAF sensor according to the present disclosure may be operable to estimate distances between about 1 millimeter to about 400 centimeters.

More particularly, the LDAF sensor may include at least one emitter and a plurality of receivers and/or collectors. It should be understood that, as used herein, the terms “receivers” and “collectors” may be used interchangeably. The at least one emitter of the LDAF sensor may transmit laser pulses, such as pulses having a wavelength in a range of about 900 nanometers to about 1000 nanometers, such as about 940 nanometers. The plurality of collectors may be an array of collectors, such as an array of single-photon avalanche diode (SPAD) imagers (hereinafter “SPAD array” and/or “collector array” and/or “array”) arranged in a grid-like pattern. For instance, in some examples, the LDAF sensor may include a collector array having at least four collectors arranged in a two-by-two grid. In some examples, the LDAF sensor may include a collector array having at least nine collectors arranged in a three-by-three grid. In some examples, the LDAF sensor may include a collector array having at least sixteen collectors arranged in a four-by-four grid. In some examples, the LDAF sensor may include a collector array having at least sixty-four collectors arranged in an eight-by-eight grid. In some examples, the LDAF sensor may include a collector array having at least 144 collectors arranged in a twelve-by-twelve array. In some examples, the LDAF sensor may include a collector array having at least 256 collectors arranged in a sixteen-by-sixteen array. In some examples, the LDAF sensor may include a collector array having at least 400 collectors arranged in a twenty-by-twenty array. It should be understood, however, that the collector array may include any suitable number of collectors arranged in any suitable array having any suitable dimensions without deviating from the scope of the present disclosure.

The emitter may transmit a plurality of laser pulses towards a subject. Depending on a variety of environmental and hardware-related factors, one or more of the laser pulses may be reflected back towards the LDAF sensor. Subsequently, the plurality of collectors may receive the scattered energy (e.g., reflected laser pulses) as echoes. The LDAF sensor may be configured to measure, inter alia, the time it takes for the laser pulses emitted from the at least one emitter to be received by the plurality of collectors. Based on this timing, the mobile computing device may calculate and determine a distance between the LDAF sensor and the subject.

An example LDAF sensor according to example embodiments of the present disclosure can have an example field of view (FOV) and an example field of illumination (FOI), respectively. As used herein, the terms “field of view” or “FOV” and “field of illumination” or “FOI” are used to describe the area over which the LDAF sensor may effectively operate. More particularly, “field of view” or “FOV” refers to the angle and/or area within which the LDAF sensor may detect a particular subject. For instance, a “wider” FOV means the LDAF sensor may be operable over a larger area, while a “narrower” FOV means the LDAF sensor may be operable over a smaller area. In some examples, the LDAF sensor according to the present disclosure may have an FOV in a range of about 45 degrees (°) to about 180 degrees (°), such as about 60 degrees (°) to about 160 degrees (°), such as about 75 degrees (°) to about 145 degrees (°), such as about 140 degrees (°).

On the other hand, “field of illumination” or “FOI” refers to the area over which the LDAF sensor provides illumination. More particularly, the FOI of the example LDAF sensor describes the coverage area illuminated by the laser pulses emitted by the emitter. The FOI for the LDAF sensor may be affected by a variety of factors, such as type and power of the emitter, hardware, optical design, distance between the emitter and target (e.g., subject), and the like.

The FOI of the example LDAF sensor can include approximately zero percent illumination in an area that corresponds to the collector exclusion zone.

The LDAF sensor may include an array of collectors arranged in a grid-like pattern. In some examples, the LDAF sensor may include a collector array having sixteen collectors arranged in a four-by-four grid. In such examples, the LDAF sensor may have sixteen “zones” (e.g., zone 0, zone 1, zone 2, . . . , zone 15). More particularly, the LDAF sensor may have four “corner zones” (e.g., zone 0, zone 3, zone 12, zone 15) and twelve “inner zones” (e.g., zones 1-2, zones 4-11, zones 13-14). Additionally and/or alternately, in some examples, the LDAF sensor may include a collector array having sixty-four collectors arranged in an eight-by-eight grid. In such examples, the LDAF sensor may have sixty-four “zones” (e.g., zone 0, zone 1,zone 2, . . . , zone 63). More particularly, the LDAF sensor may have four “corner zones” (e.g., zone 0, zone 7, zone 56, zone 63) and sixty “inner zones” (e.g., zones 1-6, zones 8-55, zones 57-62).

An emitter of the LDAF sensor may emit one or more laser pulses towards a target. One or more of the laser pulses emitted by the emitter may reflect off the target and, subsequently, may be detected by the collector array. Due to the optical design of the receiver, however, zone 0 (e.g., bottom left) of the collector array is illuminated by the top-right side of the target. Hence, the zone mapping of the collector array illuminated by the reflected laser pulses may be inverted in relation to the target itself.

It should be understood that an LDAF sensor having sixteen collectors arranged in a four-by-four array is described for ease of illustration and discussion. Those having ordinary skill in the art, using the disclosures provided herein, will appreciate that a collector array having any suitable number of collectors may be used without deviating from the scope of the present disclosure.

Example aspects of the present disclosure are directed to laser-based ambient sensing for mobile devices. As described herein, a user device, such as the mobile computing device described herein, can include a laser detect auto-focus (LDAF) sensor. The mobile computing device can perform ambient sensing of a user and/or an environment proximate to the user to provide ambient sensor data. By way of example, an LDAF sensor or other laser-based sensor of a mobile device can be used to ambient sensing including user sleep sensing (e.g., sleep detection, awake detection, REM detection, etc.), respiration sensing, user presence detection, user fall detection, relative localization/navigation (e.g., relative speed/position), device location, in-pocket detection of device, meditation, etc.

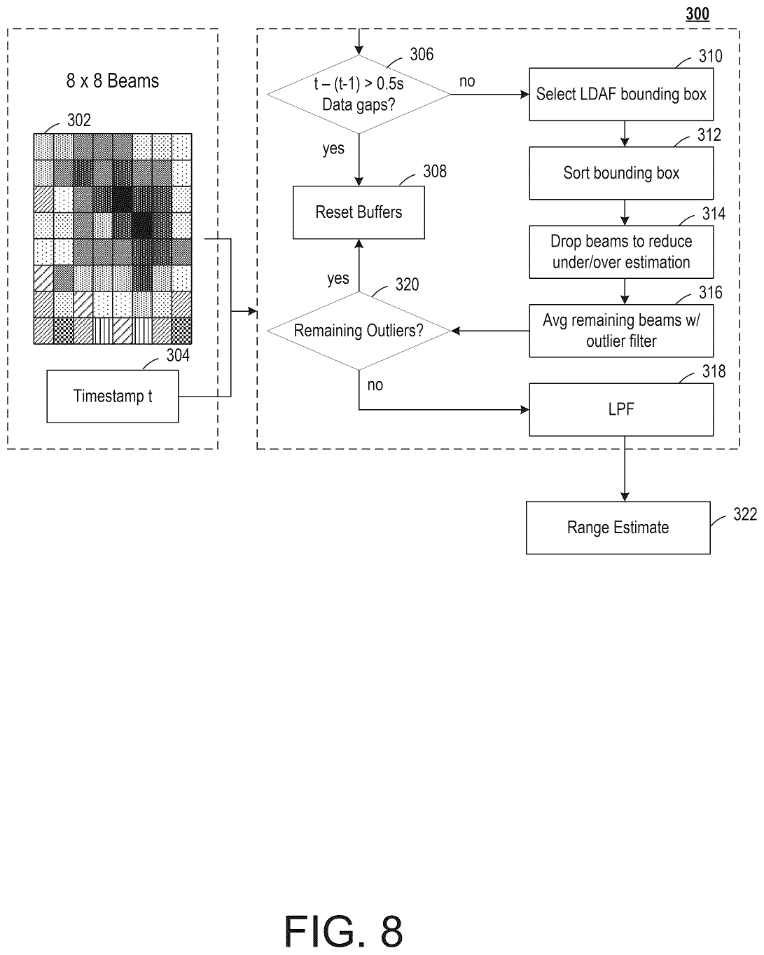

FIG. 8 depicts an example method 300 according to example embodiments of the present disclosure. According to an example aspect, an emitter of the LDAF sensor emits signals and/or beams (e.g., laser pulses) towards a reflective portion of the user's body. In some examples, the LDAF sensor emits the laser pulses towards the body of the user (e.g., a chest region of the user), to detect a respiration rate based on movement of the user's chest. According to another example, a user may hold the mobile device while the emitter of the LDAF sensor emits signals and/or beams (e.g., laser pulses) towards a fixed reflective surface (e.g., a stationary wall) to detect movement of the user relative to the fixed surface.

Accordingly, an example LDAF sensor according to the present disclosure may include a plurality of collectors arranged in a grid-like pattern (e.g., an array). For instance, in some examples, the LDAF sensor may include sixty-four collectors arranged in an eight-by-eight array 302. A timestamp 304 (t) and the reflected signals 302 can be received. At 306, the system can determine if t−(t−1) is greater than 0.5 s or another data gap. If so, the buffers can be reset at 308. If not, the system can select an LDAF bounding box at 310 and sort the bounding box at 312. At 314, the LDAF sensor may receive the reflected signals at the collector array and may drop one or more reflected beams to reduce underestimation and/or overestimation, which may reduce the likelihood of inaccurate measurements. Subsequently, the mobile computing device may calculate the average of the remaining reflected beams at 316. The system can determine if there are any remaining outliers at 320. If so, the buffers can be reset at 308. If not, the average of the remaining reflected beams can under low pass filtering at 318. The average can then be used to calculate and determine ranging information at 322. In other words, an example LDAF sensor according to the present disclosure may carefully eliminate reflected beams that would otherwise provide an invalid and/or inaccurate reading.

More particularly, the mobile computing device may eliminate one or more reflected beams that were less likely to saturate—namely, the two right-most columns in, for instance, an eight-by-eight collector array—due to those beams contributing to a variety of estimation inaccuracies. For instance, in an example where the LDAF sensor includes sixty-four collectors arranged in an eight-by-eight array, the right two columns of the collector array may greatly contribute to inaccurate and/or invalid readings. In some examples, the “max” beams may contribute to overestimation of the proximity metric, while the “min” beams may contribute to underestimation of the proximity metric. As such, the mobile computing device may discard the two right-most columns in the collector array (e.g., zones 7-8, zones 15-16, zones 23-24, zones 31-32, zones 39-40, zones 47-48, zones 55-56, zones 63-64 as shown in FIG. 15). The two right-most columns may provide the inaccurate and/or invalid readings due to its proximity to the emitter of the LDAF sensor.

Hence, the non-center beams may yield better performance. A 0.5 second smoothing filter may also be used to reduce noise variance in a range of ±1.5 millimeters. The reflected beams received by the collector array may be sorted, then dropped, and the average of the selected beams may be determined. Based on these operations, the mobile computing device may determine the range or distance.

In some examples, the LDAF sensor may have a sample rate, for instance, in a range of about 5 Hz to about 50 Hz, such as a range of about 5 Hz to about 30 Hz, such as a range of about 10 Hz to about 25 Hz, such as a range of about 12.5 Hz to about 20 Hz, such as about 15 Hz. Furthermore, in instances where the LDAF sensor includes a collector array having an eight-by-eight array of sixty-four collectors, a measurement frame may include sixty-four individual beams emitted by the emitter and received by the collector array. As shown in FIG. 8, for each frame (e.g., for each sixty-four samples), outliers (e.g., beams) may be identified, and a region of interest may be selected by dropping one or more beams that would otherwise contribute to an overestimation and/or an underestimation of the proximity metric (e.g., beams that are too far away and beams that are too close may be dropped to determine the region of interest). This outlier check and region of interest determination may be performed for each frame. After a threshold number of frames, an average may be determined. For instance, in some examples, the threshold number of frames may be in a range of about 5 frames to about 10 frames.

Furthermore, a low-pass filter (LPF) may further consider a range of about 5 frames to about 10 frames. A bounding box may then be determined and processed, and, in some examples, additional samples may be dropped from consideration. In some examples, an additional temporal check may be performed to ensure the remaining samples are sufficiently close together in time, which increases the accuracy of the determination.

In this manner, the LDAF or other laser-based sensor of a mobile computing device can be repurposed for ambient sensing. The LDAF sensor is an optical sensor that transmits pulses (e.g., 940 nm) and receives echoes to measure the distance of the observed objects. The LDAF sensor is capable of measuring (e.g., nominally) between 2.5 cnm and 400 cm depending on the hardware configuration of a particular implementation. The sensor's range precision of an observed scene can be leveraged to detect subtle range changes that can be used for applications such as respiration rate detection, stress monitoring, relative motion/localization, etc.

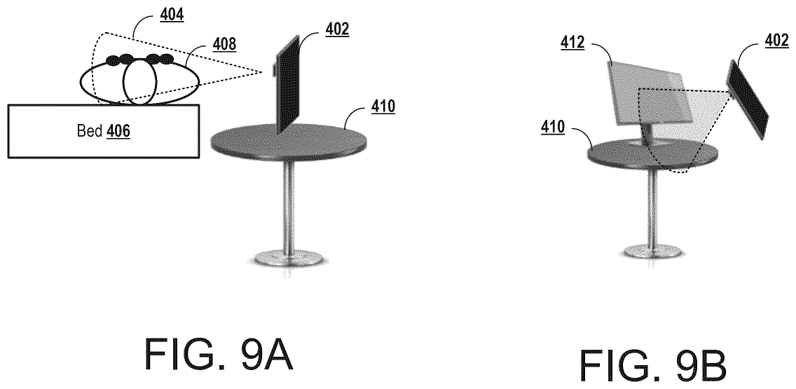

FIGS. 9A and 9B are block diagrams depicting example computing environments including a mobile computing device 402 (e.g., smartphone) having a laser-based sensor that is used for ambient sensing. FIG. 9A depicts an example where the mobile computing device 402 is placed on a stationary surface 410 such as a tripod, table, desk, etc. and is directed at a user 408 with the user within the field of view 404 of the laser-based sensor. In this example, the user is lying on a bed 406. The LDAF sensor can be used to measure, predict, or otherwise approximate the user's respiration rate. The LDAF sensor can detect distance or range changes that result from the movement of the user's chest as they breathe.

FIG. 9B depicts an example where a user holds the mobile computing device 402 while pointing the device at a stationary surface 410 such as a table or computer display 412. The LDAF sensor can detect distance or range changes that result from the movement of the user and the mobile computing device relative to the fixed surface. In either example, the LDAF sensor data can be processed using various techniques to determine the user's respiration rate. In an example embodiment, the LDAF sensor data (raw data or pre-processed data) can be provided as an input to a machine-learned model (e.g., a classification model) that has been trained to generate an output including a predicted respiration rate based on the sensor data. According to an example aspects, an LDAF sensor for respiration rate sensing can include settings such as a frame rate equal to 15 Hz, 30 Hz, or 60 Hz, single zone with 1×1 beams or multi-zone with 8×8 or 4×4 beams (or receivers), a max range of 400 cm, and a field of view (FoV) of 60 degrees. The hardware settings can be generalized to higher/lower frame rates and other beam configurations.

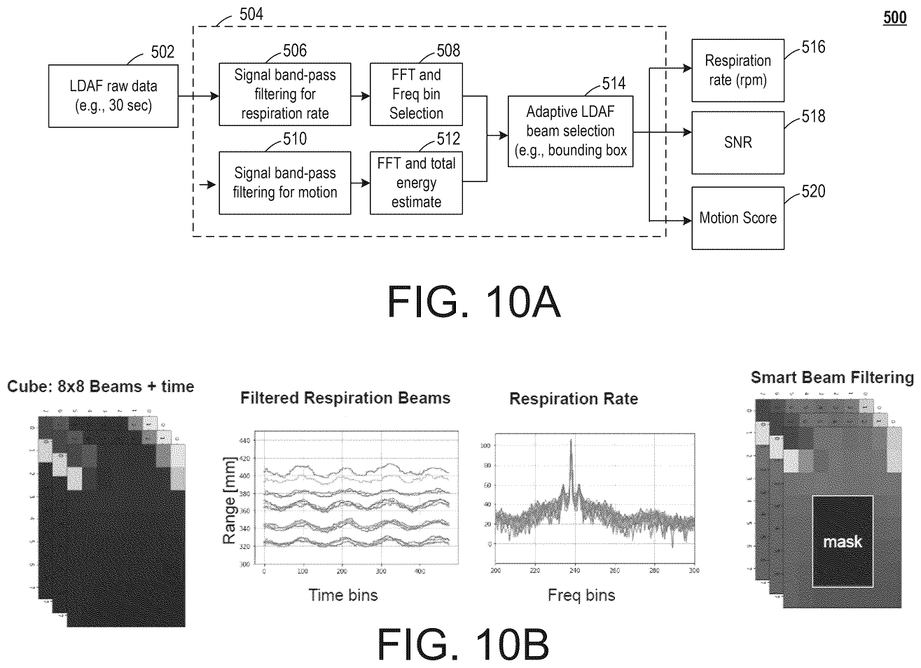

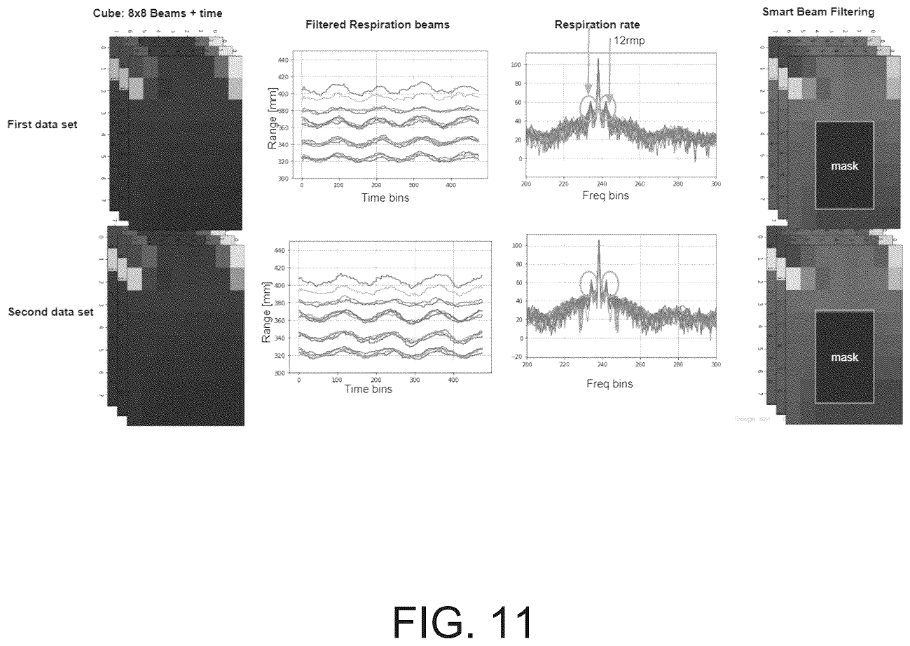

FIG. 10AA is a block diagram depicting an example of a respiration rate sensing system 500 in accordance with example embodiments of the present disclosure. FIG. 10B depicts an example of processing LDAF sensor data to determine respiration rate with the system of FIG. 10A. FIG. 10A and FIG. 10B depict an example of respiration rate sensing where the mobile computing device is stationary and is pointing at a user.

With reference to FIG. 10A, the LDAF sensor of the mobile computing device is pointing at a user and generates sensor data 502 (e.g., a sequence of sensor data measurements over a 30 second period). The raw LDAF sensor data 502 is provided to a processing pipeline 504 which operates over a sliding window of time series data. FIG. 10B depicts an example of raw sensor data including 8×8 beams over a period of time. The raw sensor data is provided in a first signal path for respiration rate determination. The raw sensor data 502 is provided to a signal band-pass filtering system 506 which filters the LDAF sensor data to select a band that contains the relevant range information for determining respiration rate. FIG. 10B depicts an example of filtered respiration beams. The filtering system can filter out unwanted frequencies which are out of the respiration rate region, which may be in the range of 19 to 25 rpms. The filtered LDAF sensor data is provided to a fast-fourier transform and frequency bin selection system 508. FIG. 10B depicts an example of the respiration data after frequency binning.

Similarly, the raw sensor data is provided to a second signal path for motion measurement. The raw sensor data is provided to a signal band-pass path filtering system 510 which filters the LDAF sensor data to select a band that contains the relevant range information for determining motion. The system can filter out unwanted frequencies in order to keep the high frequency component related to large user movements in example embodiments. The filtered LDAF sensor data is provided to a fast-fourier transform and energy estimation system 512. After transformation and binning, the data from the respiration rate signal path is provided to an adaptive LDAF beam selection system 514. Similarly, the data from the motion signal path is provided to the adaptive LDAF beam selection system 514. The system can implement a bounding box algorithm in example embodiments. The adaptive LDAF beam selection system can generate a first output 516 including the respiration rate (e.g., in revolutions per minute RPM), a second output 518 including a signal to noise ratio (SNR), and a third output 520 including a motion score.

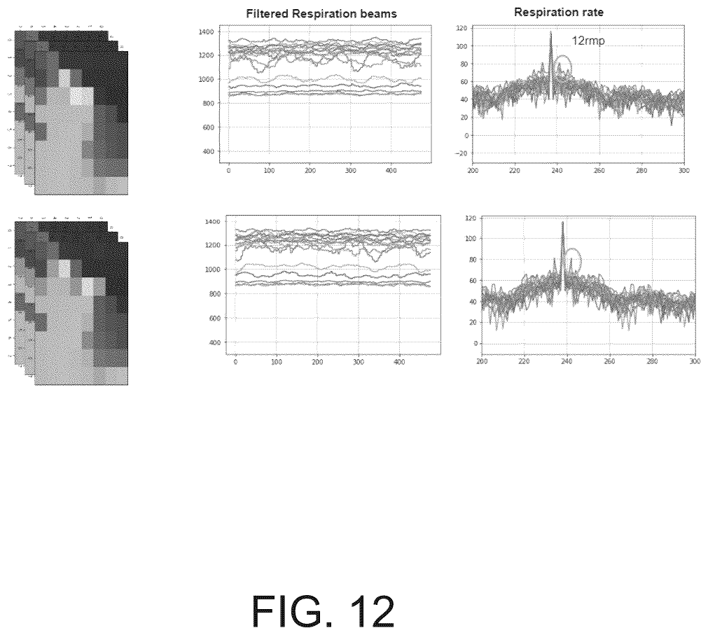

FIG. 11 depicts a graphical illustration of a first data set and a second data set including raw LDAF sensor data. The raw sensor data includes 8×8 beams over a period of time. The raw sensor data is filtered into filtered respiration beams. The filtered data is transformed and binned into frequency bins. The respiration rate is shown by peaks in the frequency bins. The motion is shown by peaks in the frequency bins. Smart beam filtering is applied to determine respiration rate, motion score, and/or the signal to noise ratio.

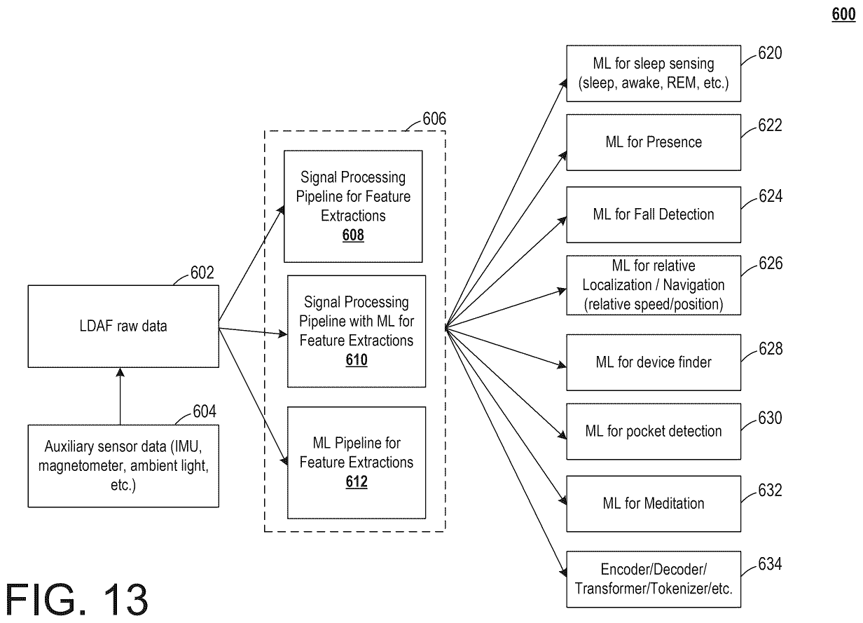

FIG. 12 depicts a graphical illustration of a first data set and a second data set including raw LDAF sensor data. FIG. 12 depicts an example where the raw sensor data is generated by the LDAF in response to the mobile computing device moving relative to a fixed surface. The raw sensor data includes 8×8 beams over a period of time. The raw sensor data is filtered into filtered respiration beams. The filtered data is transformed and binned into frequency bins. The respiration rate is shown by peaks in the frequency bins. The motion is shown by peaks in the frequency bins. Smart beam filtering is applied to determine respiration rate, motion score, and/or the signal to noise ratio. FIG. 12 demonstrates that even subtle movements can be sensed based on relative hand-held device versus scene motion to estimate respiration rate. Similarly, subtle range changes can be used to generate a very course map of the observed scene and estimate relative phone/user speed and direction for localization and/or navigation.

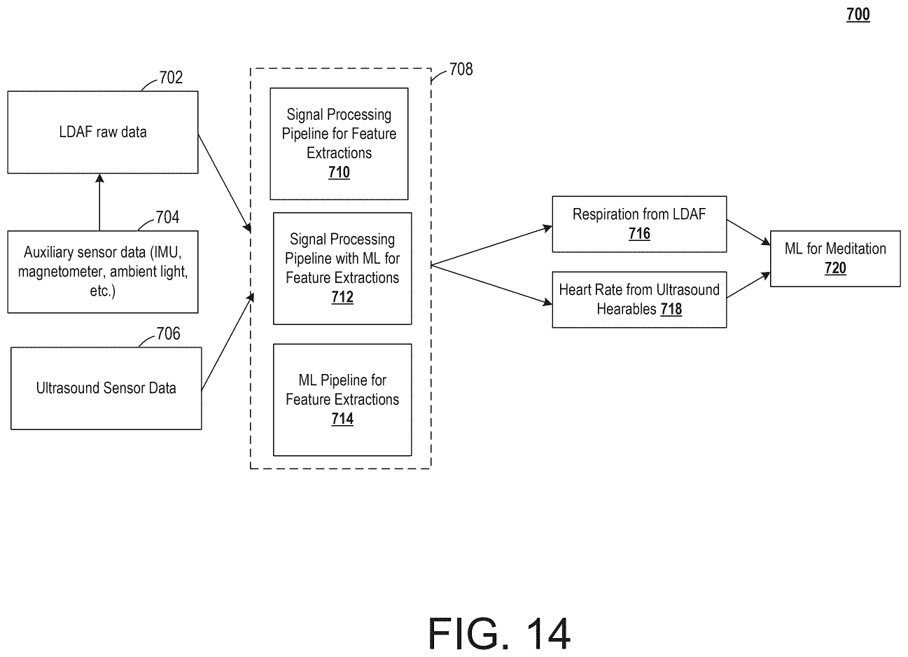

FIG. 13 is a block diagram of a computing environment 600 including a processing pipeline for processing laser-based sensor data for ambient sensing applications. Laser-based sensor data such as raw LDAF sensor data 602 can be generated by one or more LDAF or other laser-based sensors. Optionally, auxiliary sensor data 604 from one or more auxiliary sensors (e.g., inertial measurement unit (IMU), magnetometer, ambient light, etc.) can be generated. The LDAF sensor data can be provided to an optional pre-processing system 606. The pre-processing system 606 can include a signal processing pipeline 608 for feature extraction. The pipeline can be configured to extract or otherwise determine features from the raw LDAF sensor data. The pre-processing system can include a combined pipeline 610 for feature extractions that includes signal processing and one or more machine-learned models for feature extraction from the raw LDAF sensor data. The pre-processing system can include a machine-learning pipeline 612 including one or more machine-learned models for feature extractions. It is noted that the pre-processing system can include one or more of the described pipelines and/or additional pipelines. Further, the pre-processing system is optional as the raw LDAF sensor data may be processed directly by a machine-learning system in example implementations.

The pre-processed and/or the raw sensor data can be provided to a machine-learning system including one or more machine-learned models for various applications. The machine-learning system can include one or more machine-learned models 620 configured for sleep sensing. For example, one or more machine-learned classification or prediction models can be trained to predict a sleep state, an awake state, an REM sleep pattern, etc. based on the raw or pre-processed LDAF sensor data. One or more machine-learned classification or detection models 622 can be trained to detect the presence of an object or person based on the raw or pre-processed LDAF sensor data. One or more machine-learned classification or prediction models 624 can be trained to identify a fall of a user based on the raw or pre-processed LDAF sensor data. One or more machine-learned classification or prediction models 626 can be trained for relative localization/navigation prediction such as relative speed/position based on the raw or pre-processed LDAF sensor data. One or more machine-learned classification or prediction models 628 can be trained to locate a device based on the raw and/or pre-processed LDAF sensor data. One or more machine-learned classification or prediction models 630 can be trained to detect when a device is located in a pocket based on the raw and/or pre-processed LDAF sensor data. One or more machine-learned classification or prediction models 632 can be trained to determine meditative conditions based on the raw and/or pre-processed LDAF sensor data. The ML system can additionally include one or more encoders, decoders, transformers, and/or tokenizers, etc. 634 configured for other tasks such as compression or to work with LLMs.

According to an example aspect of the present disclosure, the sampling rate of the LDAF sensor can be adjusted for different use cases or applications. The sampling rate can be adjusted to generate first sensor data using a first sampling rate and second sensor data using a second sampling rate. The first sensor data can be used for a first use case and the second sensor data can be used for a second use case. For example, the first sensor data can be used to determine a first biometric of a user (e.g., respiration rate) and the second sensor data can be used to determine a second biometric of the user (e.g., sleep state or pattern). In another example, the first sensor data can be used for a first use case such as for determining a user biometric and the second sensor data can be used for a second use case such as for presence detection, localization, etc.

FIG. 14 is a block diagram of a computing environment 700 including a processing pipeline for processing laser-based sensor data for a meditation application. Laser-based sensor data such as raw LDAF sensor data 702 can be generated by one or more LDAF or other laser-based sensors. Optionally, auxiliary sensor data 704 from one or more auxiliary sensors (e.g., inertial measurement unit (IMU), magnetometer, ambient light, etc.) can be generated. Optionally, ultrasonic sensor data 706 from one or more ultrasonic sensors (e.g., speaker/microphone of a hearable device) can be generated. The sensor data can be provided to an optional pre-processing system 708. The pre-processing system can include a signal processing pipeline 710 for feature extractions that is configured to extract or otherwise determine features from the raw LDAF sensor data. The pre-processing system can include a combined pipeline 712 for feature extractions that includes signal processing and one or more machine-learned models for feature extraction from the raw LDAF sensor data. The pre-processing system can include a machine-learning pipeline 714 including one or more machine-learned models for feature extractions. It is noted that the pre-processing system can include one or more of the described pipelines and/or additional pipelines. Further, the pre-processing system is optional as the raw LDAF sensor data may be processed directly by a machine-learning system in example implementations.

The processing pipeline can include a machine-learning system including one or more machine-learned models. The system can include one or more machine-learned classification or prediction models 710 configured to determine a respiration rate 716 from the raw and/or pre-processed LDAF sensor data. The system can include one or more machine-learned classification or prediction models 712 configured to determine a heart rate 718 from the raw and/or pre-processed ultrasonic sensor data. The respiration rate and/or heart rate can be provided to a machine-learned classification or prediction model 720 configured to generate one or more meditation classifications and/or predictions.

While the present subject matter has been described in detail with respect to specific example embodiments thereof, it will be appreciated that those skilled in the art, upon attaining an understanding of the foregoing can readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A computer-implemented method, comprising:
generating, with one or more laser-based sensors of a mobile computing device over a period of time, sensor data indicative of a distance between the mobile computing device and at least one surface; and
generating, with one or more machine-learned models and based on the sensor data, ambient sensing data including at least one biometric associated with a user of the mobile computing device.

2. The computer-implemented method of claim 1, wherein:
the ambient sensing data includes at least one of presence information or localization information.

3. The computer-implemented method of claim 1, wherein:
the one or more laser-based sensors of the mobile computing device include a laser detect auto-focus sensor.

4. The computer-implemented method of claim 3, further comprising:
determining a focus setting for at least one image capture device of the mobile computing device based at least in part on sensor data generated with the one or more laser-based sensors.

5. The computer-implemented method of claim 1, wherein the at least one biometric of the user comprises a respiration rate of the user.

6. The computer-implemented method of claim 1, wherein the at least one biometric of the user comprises a heart rate of the user.

7. The method of claim 1, wherein generating, with one or more laser-based sensors of the mobile computing device over the period of time, sensor data indicative of the distance between the mobile computing device and at least one surface, comprises:
emitting, via a LDAF sensor of the mobile computing device, one or more signals in a direction towards the user;
receiving, via the LDAF sensor, one or more reflected signals; and
generating the sensor data based on the one or more reflected signals.

8. The method of claim 1, wherein generating, with one or more laser-based sensors of the mobile computing device over the period of time, sensor data indicative of the distance between the mobile computing device and at least one surface, comprises:
emitting, via a LDAF sensor of the mobile computing device while the mobile computing device is held by the user, one or more signals in a direction of a stationary surface;
receiving, via the LDAF sensor, one or more reflected signals; and
generating the sensor data based on the one or more reflected signals.

9. The method of claim 7, wherein receiving the one or more reflected signals comprises receiving, at a collector array of the LDAF sensor, the one or more reflected signals.

10. The method of claim 9, wherein the collector array comprises at least one collector.

11. The method of claim 3, wherein generating, with one or more laser-based sensors of a mobile computing device over a period of time, sensor data indicative of the distance between the mobile computing device and at least one surface, comprises:
receiving, via the LDAF sensor, a frame of reflected signals; and
determining a region of interest based on the frame of reflected signals.

12. The method of claim 1, wherein:
the ambient sensing data includes at least one of sleep sensing, presence, fall detection, localization, navigation, device finding, pocket detection, meditation, or gesture detection.

13. The method of claim 3, wherein the sensor data is indicative of a distance between the user and the LDAF sensor of the mobile computing device.

14. The method of claim 1, wherein:
generating sensor data indicative of a distance between the mobile computing device and at least one surface, comprises:
adjusting the sampling rate to generate first sensor data using a first sampling rate and second sensor data using a second sampling rate;
generating ambient sensing data including at least one biometric associated with a user of the mobile computing device comprises:
generating ambient sensing data including a first biometric based on the first sensor data generated using the first sampling rate and a second biometric based on the second sensor data generated using the second sampling rate.

15. A mobile computing device, comprising:
an image capture assembly having an image capture device and one or more laser-based sensors; and
one or more computing devices configured to perform one or more operations, the operations comprising:
generating, with the one or more laser-based over a period of time, sensor data indicative of a distance between the mobile computing device and at least one surface; and
generating, with one or more machine-learned models based on the sensor data, ambient sensing data including at least one biometric associated with a user of the mobile computing device.

16. The mobile computing device of claim 15, wherein:
the one or more laser-based sensors of the mobile computing device include a laser detect auto-focus sensor.

17. The mobile computing device of claim 15, wherein the operations further comprise:
determining a focus setting for at least one image capture device of the mobile computing device based at least in part on sensor data generated with the one or more laser-based sensors.

18. A computing system, comprising:
one or more processors; and
one or more non-transitory computer-readable media that collectively store:
one or more machine-learned models; and
instructions that, when executed by the one or more processors, cause the computing system to perform operations, the operations comprising:
generating, with one or more laser-based sensors of a mobile computing device over a period of time, sensor data indicative of a distance between the mobile computing device and at least one surface; and
generating, with the one or more machine-learned models based on the sensor data, ambient sensing data including at least one biometric associated with a user of the mobile computing device.

19. The computing system of claim 18, wherein:
the one or more laser-based sensors of the mobile computing device include a laser detect auto-focus sensor.

20. The computing system of claim 18, wherein the operations further comprise:
determining a focus setting for at least one image capture device of the mobile computing device based at least in part on sensor data generated with the one or more laser-based sensors.
